(19) United States
(12) Patent Application Publication
(10) Pub. No.: US 2023/0103644 A1
(43) Pub. Date: Apr. 6, 2023

(54) TREATMENT OF EPILEPSY

(71) Applicant: Katholieke Universiteit Leuven, Leuven (BE)

(72) Inventors: Bruno Cammue, Alsemberg (BE); Peter De Witte, Kessel-Lo (BE); Lieven Lagae, Oud-Heverlee (BE); Karin Thevissen, Bierbeek (BE)

(21) Appl. No.: 17/906,138

(22) PCT Filed: Mar. 16, 2021
(86) PCT No.: PCT/EP2021/056594

§ 371 Date: Sep. 12, 2022

(30) Foreign Application Priority Data
Mar. 16, 2020 (EP) 20163258.5

(51) Int. Cl. A61K 31/47; A61K 31/137; A61P 25/08

(52) U.S. Cl. CPC A61K 31/47; A61K 31/137; A61P 25/08

(57) ABSTRACT

The invention relates to 8-hydroxyquinolines or pharmaceutically acceptable salt thereof for use in the prevention or treatment of epilepsy.

### FIELD OF THE INVENTION

The present invention relates to the treatment of epilepsy. The present invention further relates to 8-hydroxyquinolines that show Phgdh activation.

### BACKGROUND OF THE INVENTION

The L-serine biosynthetic enzyme 3-phosphoglycerate dehydrogenase (Phgdh) is one of the enzymes implicated in de novo serine synthesis [reviewed in Grant (2018) Front Mol Biosci. 5, 110]. In human, Phgdh deficiencies have been reported; the hallmarks of Phgdh deficiency are microcephaly of prenatal onset, severe psychomotor disability, early intractable seizures (of various type), and progressive spasticity. Phgdh deficiencies can be subdivided in two severe recessive phenotypes: classical Phgdh deficiency (OMIM 601815; characterized by 12-25% residual Phgdh activity [Tabatabaie et al. (2011) J Inherit Metab Dis. 34, 181-184].

Expanding the clinical spectrum of 3-phosphoglycerate dehydrogenase deficiency. L1,] and Neu-Laxova syndrome (NLS) type 1 (OMIM 256520). In NLS1, and in the adult form, no Phgdh enzymatic activity has been reported so far. Patients reported to date show severe, early onset, drug resistant epilepsy [Poli et al. (2017) Am J Med Genet A. 173, 1936-1942]. Hence, drug resistant epilepsy might be associated with Phgdh malfunctioning/deficiency.

Yang et al. (2010) J Biol Chem. 285, 41380-41390, disclose a conditional Phgdh knockout mouse model indicating that L-serine (synthesized via Phgdh activity) is a key rate-limiting factor for maintaining steady-state levels of D-serine in adult brain. Hence, this study demonstrates that L-serine availability in mature neuronal circuits determines the rate of D-serine synthesis in the forebrain and controls NMDA receptor function at least in the hippocampus.

Sim et al. (2020) Metabolism 102, 154000, and Aksoy et al. (2014) Neurol Sci. 35, 1441-1446 disclose that mice with reduced Phgdh expression, induced by a diet resulting in development of fatty liver disease, have a severe predisposition for development of seizures (increase seizure episodes, decreased seizure thresholds. WO94/17042 discloses quinoline carboxylates such as 7-chloro-kynurenic acid as anticonvulsants.

Kynurenic acid and derivatives are antagonists of the glycine binding site in the NMDA receptor. Herein, the N-containing ring is interacting with the glycine binding site through hydrogen-bonding/accepting groups. Also the carbonyl-group at position mimicking the carboxy-group in glycine appears to contribute to the glycine binding site. The other aromatic ring is halogenated to create a hydrophobic moiety within the structure which also adds to the high affinity of the compounds for the glycine binding site.

The present invention discloses compounds that can increase the activity of Phgdh in a neurological context and are suitable in treating epilepsy, including drug resistant epilepsy.

The present invention allows to identify a cohort of patients with an epilepsy resulting from a Phgdh deficiency. Such cohort can be identified via a DL-serine assay kit (Abcam) to identify an abnormal L vs D serine content and/or Phgdh activity measurement kit to identify individuals with impaired Phgdh activity and/or Phghd expression levels.

### SUMMARY OF THE INVENTION

The present invention discloses Phgdh activators which belong to the class of haloquinolines. We have demonstrated efficacy of the haloquinolines chloroxine and clioquinol in zebrafish models for drug resistant epilepsy, including a genetic model for Dravet syndrome as well as in the 6Hz-mouse model.

Embodiments of the present invention relate to haloquinolines, and in particular clioquinol and chloroxine, for use in Dravet-associated epilepsy, and for drug resistant epilepsy in general.

The invention is further summarized in the following statements:

1. A haloquinoline or pharmaceutically acceptable salt thereof for use as neuroprotective agent.

2. The haloquinoline or salt for use according to statement 1, in the prevention or treatment of epilepsy.

3. The haloquinoline or salt for use according to statement 1 or 2, which is a 8-hydroxyquinoline.

4. The haloquinoline or salt for use according to any one of statements 1 to 3, wherein the haloquinoline is selected from the group consisting of clioquinol, chloroxine and broxyquinoline.

5. The haloquinoline or salt for use according to any one of statements 1 to 4, wherein the epilepsy is a drug resistant epilepsy.

6. The haloquinoline or salt for use according to any one of statements 1 to 5, wherein the epilepsy is a genetic disorder.

7. The haloquinoline or salt for use according to any one of statements 1 to 6, wherein the epilepsy is Dravet syndrome.

8. The haloquinoline or salt for use according to any one of statements 1 to 7, wherein the haloquinoline is administered at an amount of between 250 to 800 mg per/day.

9. The haloquinoline or salt for use according to any one of statements 1 to 8, wherein the haloquinoline is administered orally.

10. The haloquinoline or salt for use according to any one of statements 1 to 9, wherein the haloquinoline treatment is a monotherapy.

11. The haloquinoline or salt for use according to any one of statements 1 to 9, in a combination treatment with fenfluramine.

12. A 8-hydroxyquinoline or pharmaceutically acceptable salt thereof for use in the prevention or treatment of epilepsy.

13. A 8-hydroxyquinoline or pharmaceutically acceptable salt thereof for use in the prevention or treatment of epilepsy according to statement 12 wherein the 8-hydroxyquinoline is halogenated at position 5 and/or 7.

14. The 8-hydroxyquinoline or salt for use according to statement 12 or 13, wherein the 8-hydroxyquinoline is selected from the group consisting of clioquinol, chloroxine and broxyquinoline.

15. The 8-hydroxyquinoline or salt for use according to any one of statement 12 to 14, which is clioquinol.

16. The 8-hydroxyquinoline or salt for use according to any one of statements 12 to 15, wherein the 8-hydroxyquinoline is not in a complex with Zinc.

17. The 8-hydroxyquinoline or salt for use according to any one of statements 12 to 16, wherein the epilepsy is a drug resistant epilepsy.

18. The 8-hydroxyquinoline or salt for use according to any one of statements 12 to 17, wherein the drug resistant epilepsy is resistant against two or more selected from the group consisting of valproate, carbamazepine, levetiracetam, lamotrigine, topiramate, briveracetam, lacosamide, perampanel, and phenobarbital.

19. The 8-hydroxyquinolineor salt for use according to any one of statements 12 to 18, wherein the epilepsy is a genetic disorder.

20. The 8-hydroxyquinoline or salt for use according to any one of statements 12 to 19, wherein the epilepsy is Dravet syndrome.

21. The 8-hydroxyquinoline or salt for use according to any one of statements 12 to 20, in the prevention or treatment of an individual who is not from Japanese origin.

22. The 8-hydroxyquinoline or salt for use according to any one of statements 12 to 20, in the prevention or treatment of an individual who is of Caucasian origin.

23. The 8-hydroxyquinoline or salt for use according to any one of statements 12 to 22, wherein the 8-hydroxyquinoline is administered at an amount of between 250 to 800 mg per/day.

24. The 8-hydroxyquinoline or salt for use according to any one of statements 12 to 23, wherein the 8-hydroxyquinoline is administered at an amount of between 1 to 3 to mg per kg/day.

25. The 8-hydroxyquinoline or salt for use according to any one of statements 12 to 24, wherein the haloquinoline is administered orally.

26. The 8-hydroxyquinoline or salt for use according to any one of statements 12 to 25, wherein the 8-hydroxyquinoline treatment is a monotherapy.

27. The 8-hydroxyquinoline or salt for use according to any one of statements 12 to 26, in a combination treatment with a further anti-epilepsy drug.

28. The 8-hydroxyquinoline or salt for use according to any one of statements 12 to 27, in a combination treatment with fenfluramine.

29. A method of treating or preventing epilepsy in a human individual, comprising the step of administering an effective amount of a 8-hydroxyquinoline.

### DETAILED DESCRIPTION

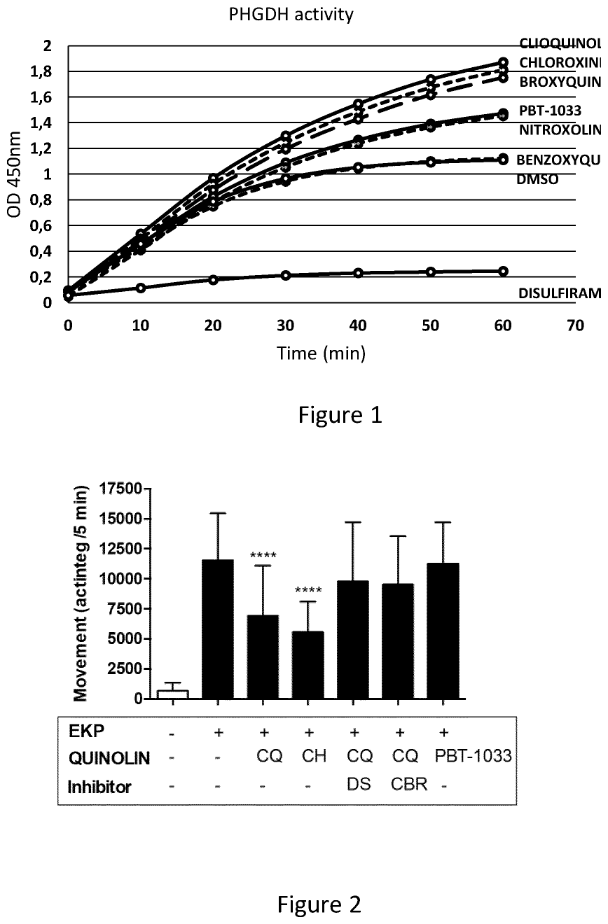

FIG. 1. Phgdh activity over time (in vitro) in presence of 25 μM of different 8-hydroxyquinolines.

FIG. 2. Activity profile of compounds in zebrafish epilepsy model; clioquinol (CQ) 3 μM, chloroxine (CH) 3.125 μM and PBT-1033 6 μM and/or inhibitors disulfiram (DS) 0.156 μM and CBR 0.0781 μM after 300 μM EKP exposure. Statistical analysis: one-way ANOVA with Dunnett's multiple comparison test. A statistical difference is indicated by: *p<0.05, **p<0.01 and ***p<0.001. for each condition n=10 larvae were used and the experiment was performed twice (n total=20 per condition).

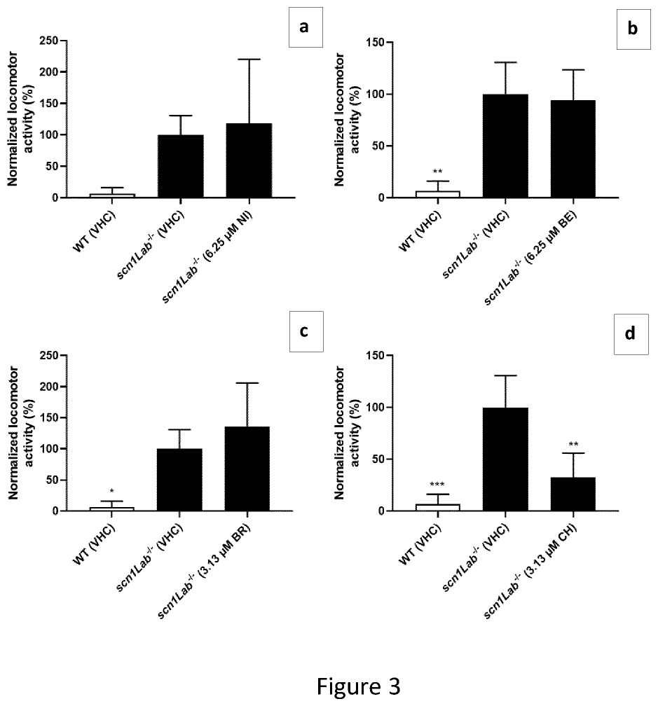

FIG. 3. Activity profile of nitroxoline (NI), benzoxyquine (BE), broxyquinolin (BR) and CH (locomotor behavioural assays). Locomotor activity was normalized against VHC-treated scn1Lab mutant larvae and displayed as a percentage±SD. Statistical analysis: one-way ANOVA with Dunnett's multiple comparison test. A statistical difference is indicated by: *p<0.05, **p<0.01 and ***p<0.001 vs. VHC-treated Scn1Lab mutant controls. n=3-4 larvae for each experimental condition.

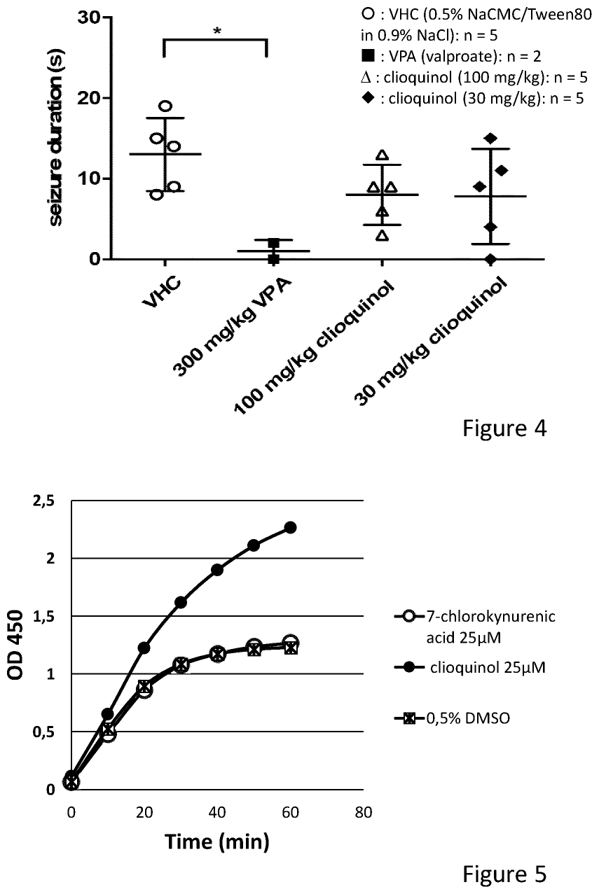

FIG. 4. Antiseizure activity analysis of clioquinol in the mouse 6-Hz (44 mA) psychomotor seizure model. Drug-resistant psychomotor seizures were induced by electrical stimulation through the cornea, 60 min after i.p. injection of vehicle (VHC, n=5), positive control valproate (n=2), or compound X (n=5). Mean seizure durations (±SD) are depicted. Statistical analysis: one-way ANOVA with Dunnett's multiple comparison test (GraphPad Prism 8). Significance levels: *p≤0.05.

FIG. 5. Phgdh activity over time in presence of 25 μM of clioquinol or 7-chloro-kynurenic acid.

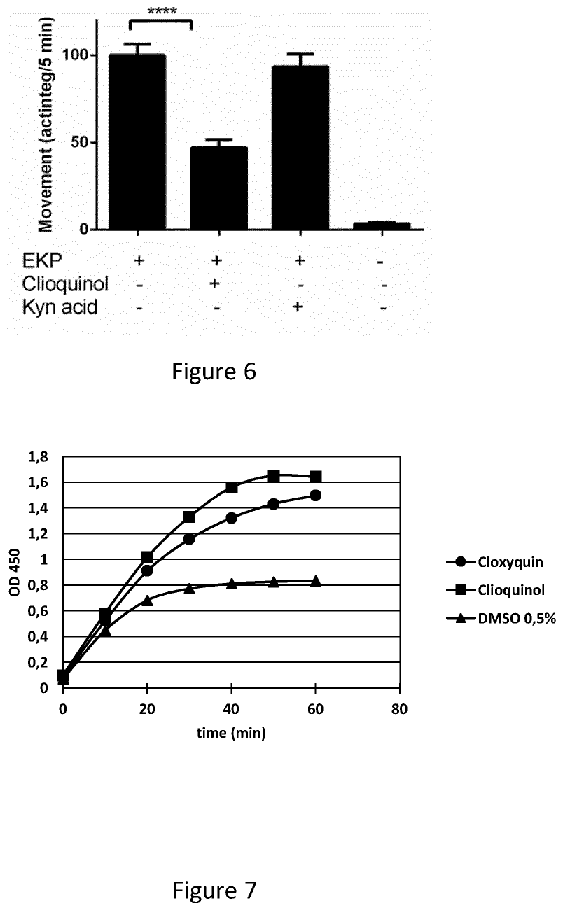

FIG. 6. Activity profile of 1 μM clioquinol and 1 μM 7-chlorokynurenic acid (Kyn acid) after 300 μM EKP exposure.

FIG. 7. Phgdh activity over time in presence of 25 μM of clioquinol or cloxyquin.

One aspect of the present invention relates to a haloquinoline for neuroprotection in a subject, whereby the haloquinoline activates Phgdh activity.

Conditions wherein “neuroprotection” prevents or treats a disorder are for example stroke, Parkinson's disease, Alzheimer's disease, Huntington's disease, multiple sclerosis, amyotrophic lateral sclerosis, AIDS-induced dementia, epilepsy, alcoholism, alcohol withdrawal, drug-induced seizure, viral/bacterial/fever-induced seizure, trauma to the head (traumatic brain injury), spinal cord injury, hypoglycaemia, hypoxia, myocardial infarction, cerebral vascular occlusion, cerebral vascular haemorrhage, haemorrhage, an environmental excitotoxin, dementia, trauma, drug-induced brain damage, stroke/ischemia, and aging.

“Seizure” refers to a brief episode of signs or symptoms due to abnormal excessive or synchronous neuronal activity in the brain. The outward effect can vary from uncontrolled jerking movement (tonic-clonic seizure) to as subtle as a momentary loss of awareness (absence seizure).

Seizure types are typically classified on observation (clinical and EEG) rather than the underlying pathophysiology or anatomy.

I Focal seizures (Older term: partial seizures)

IA Simple partial seizures—consciousness is not impaired

IA1 With motor signs

IA2 With sensory symptoms

IA3 With autonomic symptoms or signs

IA4 With psychic symptoms

IB Complex partial seizures—consciousness is impaired (Older terms: temporal lobe or psychomotor seizures)

IB1 Simple partial onset, followed by impairment of consciousness

IB2 With impairment of consciousness at onset

IC Partial seizures evolving to secondarily generalized seizures

IC1 Simple partial seizures evolving to generalized seizures

IC2 Complex partial seizures evolving to generalized seizures

IC3 Simple partial seizures evolving to complex partial seizures evolving to generalized seizures

II Generalized seizures

IIA Absence seizures (Older term: petit mal)

IIA1 Typical absence seizures

IIA2 Atypical absence seizures

IIB Myoclonic seizures

IIC Clonic seizures

IID Tonic seizures,

IIE Tonic—clonic seizures (Older term: grand mal)

IIF Atonic seizures

III Unclassified epileptic seizures

A more recent classification is published in Fisher et al. (2017) Epilepsia 58(4), 522-530.

“Epilepsy” is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of subtypes of epilepsy have been characterized and categorized. The classification and categorization system, that is widely accepted in the art, is that adopted by the International League Against Epilepsy's (“ILAE”) Commission on Classification and Terminology [See e.g., Berg et al. (2010), “Revised terminology and concepts for organization of seizures,” Epilepsia, 51(4), 676-685]:

I. Electrochemical Syndromes (Arranged by Age of Onset):
I.A. Neonatal period: Benign familial neonatal epilepsy (BFNE), Early myoclonic encephalopathy (EME); Ohtahara syndrome
I.B. Infancy: Epilepsy of infancy with migrating focal seizures; West syndrome; Myoclonic epilepsy in infancy (MEI); Benign infantile epilepsy; Benign familial infantile epilepsy; Dravet syndrome; Myoclonic encephalopathy in non-progressive disorders
I.C. Childhood: Febrile seizures plus (FS+) (can start in infancy); Panayiotopoulos syndrome; Epilepsy with myoclonic atonic (previously astatic) seizures; Benign epilepsy with centrotemporal spikes (BECTS); Autosomal-dominant nocturnal frontal lobe epilepsy (ADNFLE); Late onset childhood occipital epilepsy (Gastaut type); Epilepsy with myoclonic absences; Lennox-Gastaut syndrome; Epileptic encephalopathy with continuous spike-and-wave during sleep (CSWS), also known as Electrical Status Epilepticus during Slow Sleep (ESES); Landau-Kleffner syndrome (LKS); Childhood absence epilepsy (CAE)
I.D. Adolescence-Adult: Juvenile absence epilepsy (JAE); Juvenile myoclonic epilepsy (JME); Epilepsy with generalized tonic-clonic seizures alone; Progressive myoclonus epilepsies (PME); Autosomal dominant epilepsy with auditory features (ADEAF); Other familial temporal lobe epilepsies
I.E. Less specific age relationship: Familial focal epilepsy with variable foci (childhood to adult); Reflex epilepsies

II. Distinctive Constellations
II.A. Mesial temporal lobe epilepsy with hippocampal sclerosis (MTLE with

II.B. Rasmussen syndrome
II.C. Gelastic seizures with hypothalamic hamartoma
II.D. Hemiconvulsion-hemiplegia-epilepsy
E. Epilepsies that do not fit into any of these diagnostic categories, distinguished on the basis of presumed cause (presence or absence of a known structural or metabolic condition) or on the basis of Primary mode of seizure onset (generalized vs. focal)

III. Epilepsies Attributed to and Organized by Structural-Metabolic Causes
III. A. Malformations of cortical development (hemimegalencephaly, heterotopias, etc.)
III. B. Neurocutaneous syndromes (tuberous sclerosis complex, Sturge-Weber, etc.)
III. C. Tumour
III. D. Infection
III. E. Trauma

IV. Angioma
IV.A. Perinatal insults
IV.B. Stroke
IV.C. Other causes

V. Epilepsies of Unknown Cause
Vi. Conditions with epileptic seizures not traditionally diagnosed as forms of epilepsy per se
   VI.A. Benign neonatal seizures (BNS)
   VI.B. Febrile seizures (FS)

A more recent classification can be found in Scheffer et al. (2017) Epilepsia. 58, 512-521.

“Drug-resistant epilepsy (DRE)” is defined by Kwan et al. (2010) Epilepsia 52(6), 1069-1077, as “failure of adequate trials of two tolerated and appropriately chosen and used antiepileptic drugs (AED schedules) (whether as monotherapies or in combination) to achieve sustained seizure freedom.”

A non-exhaustive list of anti-epileptic compounds includes Paraldehyde; Stiripentol; Barbiturates (such as Phenobarbital, Methylphenobarbital, Barbexaclone; Benzodiazepines (such as Clobazam, Clonazepam, Clorazepate, Diazepam Midazolam and Lorazepam); Potassium bromide; Felbamate; Carboxamides (such as Carbamazepine Oxcarbazepine and Eslicarbazepine acetate); fatty-acids (such as valproic acid, sodium valproate, divalproex sodium, Vigabatrin, Progabide and Tiagabine); Topiramate; Hydantoins (such as Ethotoin, Phenytoin, Mephenytoin and Fosphenytoin); Oxazolidinediones (such as Paramethadione Trimethadione and Ethadione); Beclamide; Primidone; Pyrrolidines such as Brivaracetam Etiracetam Levetiracetam; Seletracetam; Succinimides (such as Ethosuximide, Phensuximide and Mesuximide); Sulfonamides (such as Acetazolamide, Sultiame Methazolamide and Zonisamide); Lamotrigine; Pheneturide; Phenacemide; Valpromide; Valnoctamide; Perampanel; Stiripentol; Pyridoxine.

In the present invention a zebrafish model is used as model for drug resistant epilepsy. The lipid-permeable glutamic acid decarboxylase (GAD)-inhibitor, Ethyl ketopentenoate (EKP), is used that induces drug-resistant seizures in zebrafish. GAD, converting glutamate into GABA, is a key enzyme in the dynamic regulation of neural network excitability. Clinical evidence has shown that lowered GAD activity is associated with several forms of epilepsy that are often treatment resistant . This EKP-induced epilepsy zebrafish model has been validated as a model for drug-resistant epilepsy and was used to demonstrate anticonvulsant activity of various anti-epileptic drugs (AEDs)

“Dravet syndrome” is a severe form of childhood epilepsy characterized by drug-resistant seizures and numerous physical, behavioural and intellectual comorbidities. Nearly 90% of all patients with Dravet syndrome carries a mutation in the SCN1A gene (sodium channel, voltage gated, type 1 alpha subunit).

“haloquinoline” as used in the present invention relates halogenated quinolines, typically quinolines with halogen groups at position 5 and 7.

Most particular it related to halogenated 8-hydroxyquinoline as depicted in formula I or pharmaceutically acceptable salts thereof:

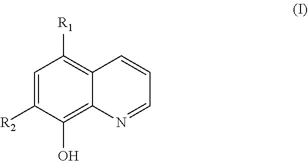

Herein R₁ and R₂ are a halogen, or R₁ is a halogen and R₂ is H, or R₁ is H and R₂ is a halogen.

Typically in dihalogenated compounds, R₁ and R₂ are each independently selected from the group consisting of fluorine (F), chlorine (Cl), bromine (Br), iodine (I).

More typically R₁ and R₂ are each independently selected from the group consisting of chlorine (Cl) iodine (I).

In specific embodiments at least one of R₁ or R₂ is chlorine.

In other specific embodiments at R₁ is chlorine.

In specific embodiments at least one of R₁ or R₂ is bromine.

In preferred haloquinolines in the context of the present invention R₁═Cl and R₂ is I (Clioquinol), or R₁═Cl and R₂═Cl (choroxine), R₁═Br and R₂═Br (broxyquinoline), or R₁═Cl and R₂ is H (cloxyquin)

Clioquinol was considered safe and efficacious for many years. It was used as an antifungal and an antiprotozoal drug until it was linked to an outbreak of subacutemyelo-optic neuropathy (SMON), a debilitating disease almost exclusively confined to Japan.

Clioquinol is much less metabolized to form conjugates in humans than in rodents. In rats, clioquinol is rapidly absorbed and undergoes first-pass metabolism to glucuronate and sulphate conjugates: the metabolites therefore reach higher concentrations than those of free clioquinol. Similar results have also been obtained in mice and rabbits, whereas monkeys, dogs, and man form much lower concentrations of metabolites and free clioquinol concentrations are higher than those of the metabolites. Dose/concentration ratios in hamster are similar to those found in other rodents, but much lower than those found in humans: at doses of 250 or 500 mg the dose/concentration ratio in humans is 0.64-1.4, which means that humans have a mean of 33 times the concentrations of free clioquinol found in hamsters (Bareggi & Cornelli (2012) CNS Neurosci Ther. 18, 41-46).

Neurological toxicity has been observed in dogs when treated with clioquinol at doses exceeding 200 mg/(kg day) for over a month, and at doses of 400 mg/(kg day) toxicity can be observed in a week (reviewed in Mao (2008) Toxicol Lett. 182, 1-6. In all but one study, a dose of 100 mg/(kg day) for over 30 days did not produced toxicity in animals.

Humans have been treated with Clioquinol at a usual dose of 1.5-2 g/day (˜25-30 mg/(kg day)), with reports of patients receiving 3.5 g (˜50 mg/(kg day))/day without toxicity. In Japanese humans, administration of daily Clioquinol has been associated with neurological side effects. Recently, evidence was gathered that clioquinol inhibits cAMP-transporting ABC pumps (reviewed in Perez et al. (2019) Pharmacol Ther. 199, 155-163). A key concept is the ability of clioquinol to block cAMP efflux from cells and thus, to trigger the phosphorylation of CREB Ser133, a classical cAMP effector that activates target genes. This finding provided a connection to possible targets of clioquinol: ABCC4 and ABCC11, transporters that normally efflux numerous endogenous substrates, including cAMP. A further analysis revealed the presence of SNPs in both ABCC4 and ABCC11, capable of reducing transporter function and at the same time present with a high frequency in the Japanese population. Modern studies showed that these SNPs are critical for patient sensitivity to cancer and immunosuppressor nucleotide-like drugs, substrates of ABCC4 and ABCC11 transporters. Thus, this line of research provides a plausible explanation for the SMON phenomenon: patients that carry SNPs in ABC transporters that dramatically affect nucleotide efflux are expected to be more sensitive to clioquinol. Since these SNPs are geographically restricted to Japan, this also accounts for the specific distribution of the disease.

Based on a recent clinical trial (Ritchie et al. (2003) Arch Neurol. 60, 1685-1691. in which the use of clioquinol was assessed against Alzheimer, no adverse effects were apparent when using 750 mg/day for weeks (dose of 10 mg/kg/day). Moreover, dose-limiting neurotoxicity and abdominal pain were observed at a dose of 3200 mg/day (dose of 42.5 mg/kg/day).

The present invention provides a zebrafish model with a mutation in the orthologous SCN1A gene (scn1Lab) which has been validated in the epilepsy field to for understanding the pathogenesis and anti-epileptic drug (AED) discovery.

The present invention discloses the effect of haloquinolines using this zebrafish line indicative of a beneficial effect on patients suffering from Dravet syndrome.

In specific embodiments the claimed use is for the treatment of epilepsy in an individual of Caucasian origin.

In specific embodiments the claimed use is for the treatment of epilepsy in an individual of who is not from Japanese origin.

### Example 1. Phgdh Activity Assay

Phgdh enzyme activity upon drug treatment was tested using human Phgdh (BPS bioscience, 71079) and a specific colorimetric Phgdh activity kit (Biovision, K569). The Phgdh inhibitor Disulfiram [Spillier et al. (2019) Sci Rep. 9, 4737], served as negative control.

Briefly, Human Phgdh enzyme was diluted in assay buffer at a concentration of 0.38 mg/mL. Next, 5 μl of this Phgdh enzyme solution and 5 μL of the compounds in 5% DMSO and assay buffer was added (to wells of flat bottom 96-well plate). Subsequently, 40 μl Phgdh assay buffer (Biovision) and 50 μl Phgdh reaction mix (prepared as described in the protocol of the Phgdh assay kit K569, Biovision) was added. The final amount of enzyme per reaction was 0.35 μM. Afterwards, absorbance at 450 nm, indicative for the amount of NADH generated, was measured over time. NADH is one of the end products of the reaction. In between measurements, the plate was incubated at 37° C., protected from light.

Activation of Phgdh was specific for the halo-quinolines clioquinol, chloroxine, broxyquinoline, and cloxyquin (resulting in 70-80% increased Phgdh activity as compared to DMSO control); other 8-hydroxyquinolines like benzoxyquine 7-chloro-kynurenic acid could not activate Phgdh (FIGS. 1 and 5).

Nitroxoline and the anti-Alzheimer clioquinol successor PBT-1033 (former PBT-2; Lanza et al. (2018) Curr Med Chem. 25, 525-539.) induced Phgdh activation (30-40% increased Phgdh activity as compared to DMSO control) albeit to a lower extent than clioquinol. Clioquinol itself does not affect the oxido-reduction reaction toward NADH generation (data not shown).

Next we tested whether 25 μM of other anti-epilepsy preclinical and clinical phase drugs as well as drugs that are on the market activate Phgdh. An overview of these drugs can be found in Table 1. None of these drugs activated Phgdh. Hence, indicating that Phgdh is a haloquinoline-specific phenomenon.

TABLE 1
Assessment of drugs that are in (pre)clinical phase or commercially
available to treat epilepsy for potential Phgdh activation

| Acetazolamide | 100 | mM | Alfa Aezer |
|---|---|---|---|
| Carbamazepine | 100 | mM | Sigma |
| Diazepam | 16 | mM | KU Leuven pharmacy |
| Eslicarbazepine | 100 | mM | TCI Europe |
| acetate | | | |
| Ethosuximide | 100 | mM | Sigma |
| Felbamate | 100 | mM | Bio-Connect |
| Gabapentin | 833 mM in Danieau's | | TCI Europe |
| Lacosamide | 100 | mM | EDQM |
| Lamotrigine | 50 | mM | TCI Europe |
| Levetiracetam | 100 | mM | Sigma |
| Oxcarbazepine | 100 | mM | TCI Europe |
| Perampanel | 100 | mM | Bio-Connect |
| Phenobarbital | 100 | mM | Fagron/KU Leuven |
| | | | pharmacy |
| Phenytoin | 100 | mM | Arcos organics/ |
| | | | Fisher scientific |
| Pregabalin | 125 mM in Danieau's | | J1K Scientific |
| Primidone | 100 | mM | Sigma |
| Rufinamide | 100 | mM | Sigma |
| Sodium | 100 mM in Danieau's/ | | Sigma |
| valproate | 50 mM in DMSO | | |
| Stiripentol | 100 | mM | TCI Europe |
| Tiagabine | 100 | mM | Sigma |
| Topiramate | 100 | mM | TCI Europe |
| Vigabatrin | 250 mM in Danieau's | | Sigma |
| Zonisamide | 100 | mM | TCI Europe |
| Fenfluramine | 20 | mM | |

all others are dissolved
in 100% DMSO

The co-administration of the Phgdh blocker disulfiram with the haloquinolines completely blocked the haloquinoline-induced Phgdh activation (data not shown).

### Example 2. Haloquinolines Block Seizures in Zebrafish Models for Drug-Resistant Epilepsy (Including a Genetic Model for Dravet)

a) Zebrafish Drug Resistant Model: Ethyl Ketopentenoate (EKP)

Glutamic acid decarboxylase (GAD) which converts glutamate into GABA is a key enzyme in the dynamic regulation of neural network excitability. Clinical evidence have shown that lowered GAD activity is associated with several forms of epilepsy which are often treatment resistant. Ethyl ketopentenoate (EKP), is a lipid-permeable GAD-inhibitor that induces drug-resistant seizures in zebrafish [Zhang et al. (2017) Sci Rep. 7, 7195]. This model can hence be used to find novel drugs to target refractory epilepsies.

Larvae (7 dpf) in 100 μl VHC were arrayed individually in a 96-well plate (tissue culture plate, at bottom, Falcon, USA) and kept in the light at 28° C. Two-hours before tracking compound (clioquinol (CQ), chloroxine (CH), PBT-1033) and/or inhibitor (disulfiram (DS), CBR) was added at their MTC to the larvae and afterwards the 96-well plates were placed in darkness at 28° C. for 2 hours. Just prior to tracking 100 p1 of VHC or EKP stock solution was added to each well to obtain a EKP concentration of 300 μM. The plates were placed in an automated video tracking device (ZebraBox™ apparatus; Viewpoint, Lyon, France) and the locomotor behavior of the larvae was monitored for 40 min in the dark at 28° C. Locomotor activity was quantified using ZebraLab™ software (Viewpoint, Lyon, France) and expressed in “actinteg” units per 5-min interval. For each larvae, 30 min of tracking data after the effect of EKP was initiated was used. The actinteg value is defined as the sum of all image pixel changes detected during the time window.

Results show that clioquinol and chloroxine significantly reduced EKP induced seizures while this effect is counteracted when co-incubated with inhibitors disulfiram and CBR (FIG. 2). These data indicate that the anti-seizure activity of clioquinol and chloroxine is Phgdh-mediated. Similar to PBT-1033, the other ‘intermediate’ Phgdh activator Nitroxoline nor the inactive compound Benzoxyquine resulted in significantly reduced movement of the zebrafish challenged with EKP in the above described setup (data not shown). Hence, this indicates that there is a minimal threshold for Phgdh activation based on the enzyme assay needed (>40% increase in Phgdh activity) to significantly reduce EKP induced seizures in vivo.

b) Zebrafish Model of Dravet Syndrome

6 dpf Scn1Lab mutant larvae (selected by their darker appearance, lack of a swim bladder and slight curvature of the body on 6 dpf) and WT larvae were arrayed in a 96-well plate (one larva per well) and treated with 100 μl VHC (0.1% DMSO) or compound (MTC concentration in 0.1% DMSO). After incubation at 28° C. on a 14/10 h light/dark cycle for 2 h, the plates were immediately placed in an enclosed tracking device (ZebraBox Viewpoint, France) and chamber habituation for 30 min. The locomotor activity of the larvae were evaluated during 10 min under dark conditions and quantified by the lardist parameter (total distance in large movements) and plotted in cm (ZebraLab™ software, Viewpoint, Lyon, France). Data was collected from 3-4 larvae per treatment condition. The locomotor data were analysed by normalizing the locomotor activity of treated larvae against VHC-treated Scn1Lab mutant larvae.

As illustrated by the FIG. 3 (a-d), Scn1Lab mutant could induce a higher locomotor activity when comparing with the WT control. Only compound CH significantly (>60% reduction) counteracted epileptiform locomotor activity of Scn1Lab mutant group and showed a good antiepileptic activity (Figure d).

### Example 3. Clioquinol can Block Seizures in the 6 Hz Mouse Model

Male NMRI mice (weight 25-30 g) were acquired from Charles River Laboratories (France) and housed in polyacrylic cages under a 14/10-hour light/dark cycle at 21° C. The animals were fed a pellet diet and water ad libitum, and were allowed to acclimate for 1 week before experimental procedures were conducted. Prior to the experiment, mice were isolated in polyacrylic cages with a pellet diet and water ad libitum for habituation overnight in the experimental room, to minimize stress. The antiseizure activity of compounds was investigated in the mouse 6-Hz (44 mA) psychomotor seizure model as described before [Copmans et al (2018) ACS Chem Neurosci. 9, 1652-1662.]. In brief, 500 μl (injection volume was adjusted to the individual weight) of VHC (0.5% sodiumcarboxymethylcellulose (NaCMC)/Tween80 in 0.9% NaCl) or treatment (valproate or clioquinol dissolved in VHC) was i.p. injected in NMRI mice and after 60 min psychomotor seizures were induced by corneal electrical stimulation (6 Hz, 0.2 ms rectangular pulse width, 3 s duration, 44 mA) using an ECT Unit 5780 (Ugo Basile, Comerio, Italy). Seizure durations were measured during the experiment by experienced researchers, familiar with the different seizure behaviours. In addition, seizure durations were determined by blinded video analysis to confirm or correct the initial observations. Data are expressed as mean±SD. Results show a trend towards reduced seizure duration in clioquinol treated animals (see FIG. 4).

### Example 4. 7-Chloro-Kynurenic Acid does not Activate PHGDH

7-chloro-kynurenic acid has been tested in the above described in vitro PHGDH enzyme assay and provokes no induced activation of PHGDH, in contrast to clioquinol (FIG. 1). This indicates that different quinolines may have with different mode of actions. Kynurenic acid and derivatives are antagonists of the glycine binding site in the NMDA receptor. Herein, the N-containing ring is interacting with the glycine binding site through hydrogen-bonding/accepting groups. Also the carbonyl-group at position mimicking the carboxy-group in glycine appears to contribute to the glycine binding site. The other aromatic ring is halogenated to create a hydrophobic moiety within the structure which also adds to the high affinity of the compounds for the glycine binding site.

### Example 4. 7-Chloro-Kynurenic Acid is not Active in an EKP-Zebrafish Model

7-chloro-kynurenic acid was equally tested in the above described EKP-zebrafish model.

Locomotor activity was quantified using ZebraLab™ software (Viewpoint, Lyon, France) and expressed in mean “actinteg” units per 5-min+/−SEM during a 30 min recording interval relative to EKP only. Statistical analysis: one-way ANOVA with Dunnett's multiple comparison test. A statistical difference is indicated by: ****p<0.0001. For each condition n=10 larvae were used and the experiment was performed two times (n total=20 per condition).

The results show a superior activity for clioquinol compared to 7-chloro-kynurenic acid, described in the prior art as an anticonvulsant agent.

### Example 5. Cloxyquin Activates PHGDH

Cloxyquin (5-Chloro-8-quinolinol) has been tested in the same conditions as in the above described in vitro PHGDH enzyme assay (example 1). FIG. 7 shows that cloxyquin has an activity comparable to clioquinol.

1-17. (canceled)

18. A method of treating epilepsy in a human individual, the method comprising:
administering to the human individual a therapeutically effective amount of an 8-hydroxyquinoline or a pharmaceutically acceptable salt thereof.

19. The method according to claim 18, wherein the 8-hydroxyquinoline is halogenated at position 5 and/or at position 7.

20. The method according to claim 18, wherein the 8-hydroxyquinoline is selected from the group consisting of clioquinol, chloroxine, and broxyquinoline.

21. The method according to claim 18, wherein the 8-hydroxyquinoline is clioquinol.

22. The method according to claim 18, wherein the 8-hydroxyquinoline is not in a complex with zinc.

23. The method according to claim 18, wherein the epilepsy is a drug-resistant epilepsy.

24. The method according to claim 23, wherein the drug resistant epilepsy is resistant against two or more drugs selected from the group consisting of valproate, carbamazepine, levetiracetam, lamotrigine, topiramate, briveracetam, lacosamide, perampanel, and phenobarbital.

25. The method according to claim 18, wherein the epilepsy is a genetic disorder.

26. The method according to claim 18, wherein the epilepsy is Dravet syndrome.

27. The method according to claim 18, wherein the human individual is not of Japanese origin.

28. The method according to claim 18, wherein the human individual is of Caucasian origin.

29. The method according to claim 18, wherein the 8-hydroxyquinoline is administered at an amount from 250 mg per day to 800 mg per day.

30. The method according to claim 18, wherein the 8-hydroxyquinoline is administered at an amount from 1 mg/kg per day to 3 mg/kg per day.

31. The method according to claim 18, wherein the 8-hydroxyquinoline is administered orally.

32. The method according to claim 18, wherein the 8-hydroxyquinoline is administered as a monotherapy.

33. The method according to claim 18, in a combination treatment with a further anti-epilepsy drug.

34. The method according to claim 18, in a combination treatment with fenfluramine.
